(19) United States
(12) Patent Application Publication
(10) Pub. No.: US 2017/0156946 A1
(43) Pub. Date: Jun. 8, 2017

(54) SANITARY NAPKIN

(71) Applicant: Yuan-Cheng CHIEN, Kaohsiung (TW)

(72) Inventor: Yuan-Cheng CHIEN, Kaohsiung (TW)

(73) Assignee: Yuan-Cheng Chien, Kaohsiung (TW)

(21) Appl. No.: 15/194,786

(22) Filed: Jun. 28, 2016

(30) Foreign Application Priority Data
Dec. 7, 2015 (TW) 104140895

(51) Int. Cl. A61F 13/531; A61F 13/15; A61F 13/47

(52) U.S. Cl. CPC A61F 13/531; A61F 13/47; A61F 13/15203; A61F 2013/4708; A61F 2013/530029

(57) ABSTRACT

A sanitary napkin includes: a substrate having a base layer and an absorbent body mounted on the base layer, and defining a longitudinal direction (X) and a width direction (Y) perpendicular to the longitudinal direction (X); and at least one absorbing unit including a plurality of connecting portions connected to the absorbent body and spaced apart among one another in the longitudinal direction (X), an absorbing portion extending in the longitudinal direction (X) and connected to the connecting portions, and a plurality of cotton fluffs disposed on the absorbing portion. The absorbent body, the connecting portions and the absorbing portion cooperatively define a plurality of aerated channels extending in the width direction (Y).

### CROSS-REFERENCE TO RELATED APPLICATION

This application claims priority of Taiwanese Patent Application No. 104140895, filed on Dec. 7, 2015.

### FIELD

The disclosure relates to a feminine hygiene product, more particularly to a sanitary napkin.

### BACKGROUND

Referring to FIG. 1, a conventional sanitary napkin 1 is shown to include a substrate 11 formed with two side flaps 111, and an absorbent body 12 mounted protrudingly on the substrate 11.

Use of the absorbent body 12 mounted protrudingly on the substrate 11 allows the sanitary napkin 1 to fittingly contact a user's skin so as to absorb secretions quickly and to prevent leakage of secretions from the sanitary napkin. Since the absorbent body 12 is in close contact with the substrate 11, and when used, the absorbent body 12 also fits closely against the user's skin, the sanitary napkin 1 thus has poor air permeability. Use of the conventional sanitary napkin for a long period of time may cause strange odors to be emitted, and comfort of use to be adversely affected.

### SUMMARY

Therefore, an object of the disclosure is to provide a sanitary napkin that can alleviate at least one of the drawbacks of the prior art.

According to the disclosure, the sanitary napkin includes: a substrate having a base layer and an absorbent body mounted protrudingly on the base layer, and defining a longitudinal direction and a width direction perpendicular to the longitudinal direction; and at least one absorbing unit including a plurality of connecting portions connected to the absorbent body of the substrate and spaced apart among one another in the longitudinal direction, an absorbing portion extending along the longitudinal direction and connected to the connecting portions, and a plurality of cotton fluffs disposed on the absorbing portion. The absorbent body of the substrate, the connecting portions and the absorbing portion cooperatively define a plurality of aerated channels each extending along the width direction.

### BRIEF DESCRIPTION OF THE DRAWINGS

Other features and advantages of the disclosure will become apparent in the following detailed description of the embodiments with reference to the accompanying drawings, of which:

FIG. 1 is a perspective view illustrating a conventional sanitary napkin;

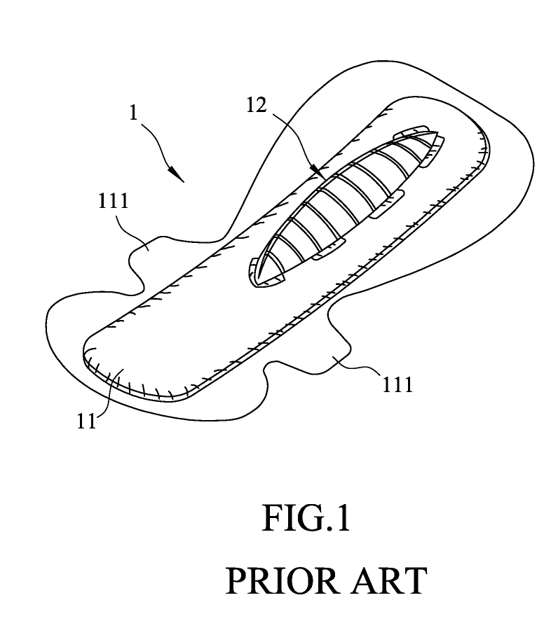

FIG. 2 is a perspective view illustrating a first embodiment of a sanitary napkin according to the disclosure;

FIG. 3 is a side view of the first embodiment;

FIG. 4 is a sectional side view of the first embodiment;

FIG. 5 is a perspective view illustrating a variation of the first embodiment;

FIG. 6 is a side view illustrating the variation of the first embodiment;

FIG. 7 is a sectional side view illustrating the variation of the first embodiment;

FIG. 8 is a perspective view illustrating a second embodiment of a sanitary napkin according to the present disclosure;

FIG. 9 is a top view illustrating a third embodiment of a sanitary napkin according to the disclosure;

FIG. 10 is a side view of the third embodiment;

FIG. 11 is a perspective view illustrating a fourth embodiment of a sanitary napkin according to the disclosure; and

FIG. 12 is a front view of the fourth embodiment.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

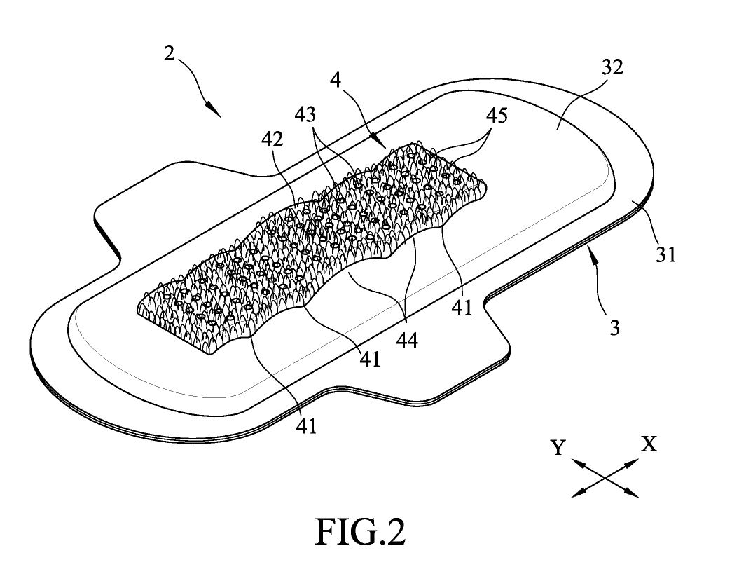

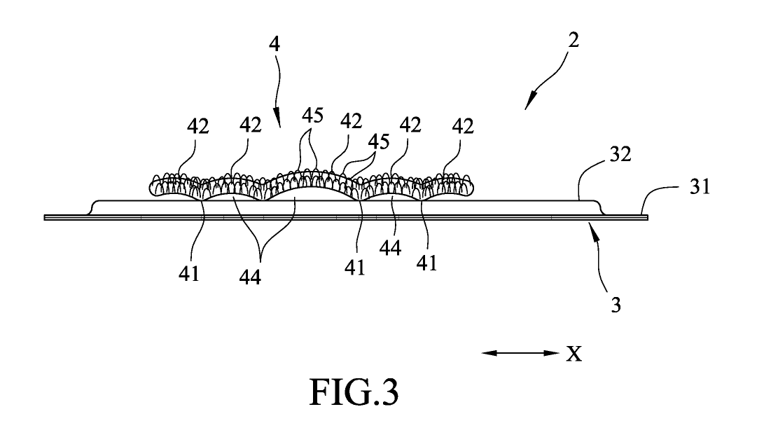

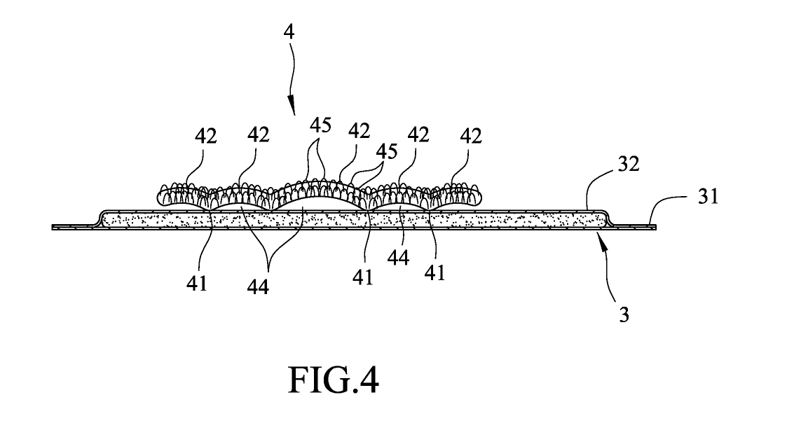

Referring to FIGS. 2 to 4, a first embodiment of a sanitary napkin 2 according to the disclosure is shown to include a substrate 3 and an absorbing unit 4 which is in the form of a sheet.

The substrate 3 has a base layer 31 and an absorbent body 32 mounted protrudingly on the base layer 31, and defines a longitudinal direction (X) and a width direction (Y) perpendicular to the longitudinal direction (X).

The absorbing unit 4 includes a plurality of connecting portions 41 connected to the absorbent body 32 of the substrate 3 and spaced apart among one another in the longitudinal direction (X), an absorbing portion 42 extending along the longitudinal direction (X) and connected to the connecting portions 41, and a plurality of cotton fluffs 45 disposed on the absorbing portion 42. In other words, the absorbing portion 42 is connected to the absorbent body 32 of the substrate 3 through the connecting portions 41. The absorbing portion 42 may be formed as parts that are each arcuate in shape. The absorbent body 32 of the substrate 3, the connecting portions 41 and the absorbing portion 42 cooperatively define a plurality of aerated channels 44 each extending along the width direction (Y).

The absorbing unit 4 is formed with a plurality of perforations 43, each spaced apart among one another, extending through the absorbing portion 42, and disposed in fluid communication with the aerated channels 44. In other words, a plurality of the perforations 43 extend through the absorbing portion 42 and are disposed in fluid communication with the aerated channels 44 that are formed under the absorbing portion 42.

It should be noted that two opposite ends of the absorbing unit 4 in the longitudinal direction (X) may be connected to or spaced apart from the absorbent body 32. In this embodiment, the two opposite ends of the absorbing unit 4 are spaced apart from the absorbent body 32.

In this embodiment, the absorbing portion 42 and the plurality of cotton fluffs 45 disposed on the absorbing portion 42 provide the effects of absorption and diffusion of the secretions, and the perforations 43 and the aerated channels 44 improve air-permeability of the sanitary napkin 2 so as to prevent the generation of strange odors and to promote comfort of use.

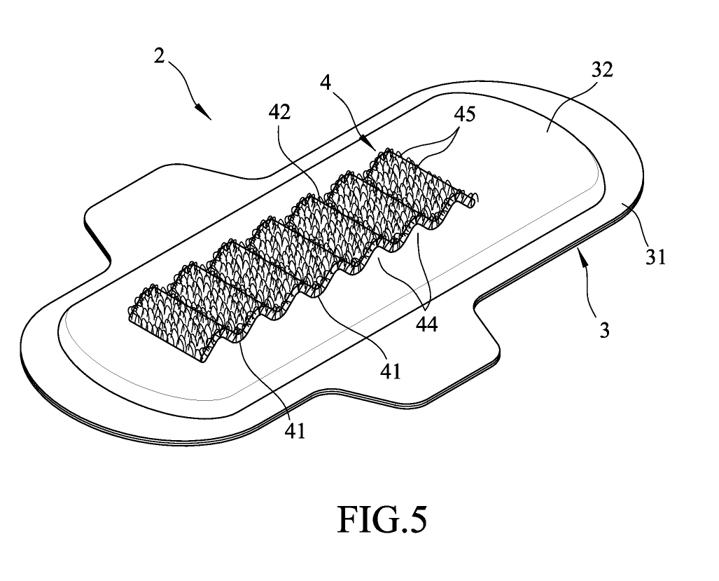

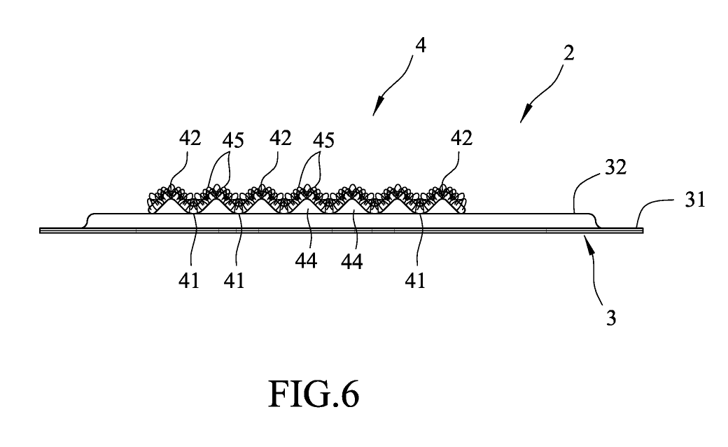

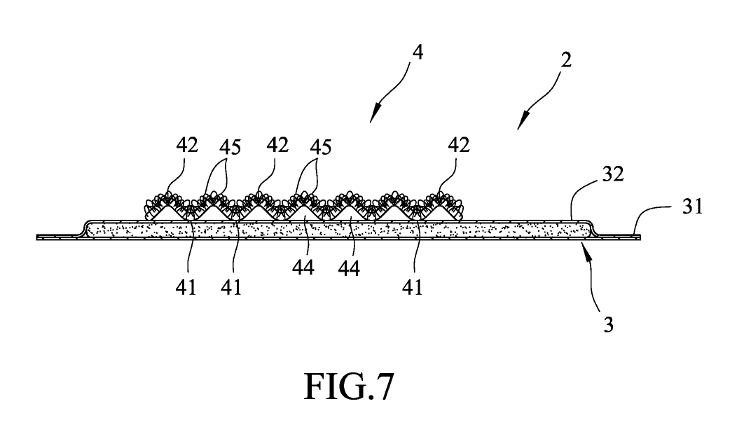

Referring to FIGS. 5 to 7, a variation of the first embodiment is shown to be nearly identical to the first embodiment, with the difference being that the absorbing portion 42 extends along the longitudinal direction in the shape of a wave, with peaks protruding away from the absorbent body 32 of the substrate 3 and valleys indented toward the absorbent body 32 of the substrate 3. In this variation of the first embodiment, FIGS. 6 and 7 illustrate that the two opposite ends of the absorbing unit 4 are connected to the absorbent body 32.

The absorbing unit 4 may further include a plurality of rows of cotton fluff balls (not shown) disposed on the absorbing portion 42, and the diameters of the cotton fluff balls in one of the rows are different from the diameters of the cotton fluff balls in an adjacent one of the rows.

The cotton fluff balls promote the effect of absorption and diffusion of the secretions. Moreover, since the diameters of the cotton fluff balls in any two adjacent rows are different, there are many gaps among the cotton fluff balls 46, which improve air permeability so as to prevent the generation of strange odors and thereby promote comfort of use.

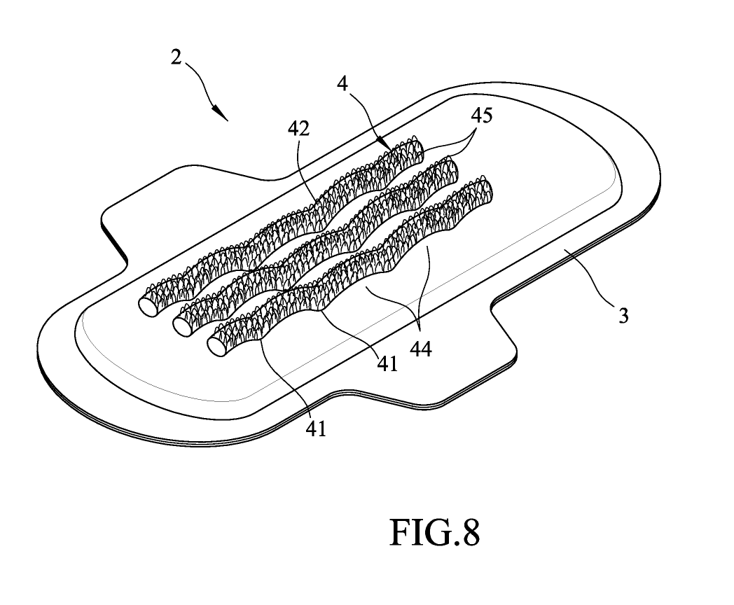

Referring to FIG. 8, a second embodiment of a sanitary napkin 2 according to this disclosure includes a substrate 3 and a plurality of absorbing units 4 that are spaced among one another along the width direction (Y). Each of the absorbing units 4 has an elongated strip shape and extends along the longitudinal direction (X).

In the second embodiment, each of the absorbing units includes a plurality of connecting portions 41 connected to the absorbent body 32 of the substrate 3 and spaced apart among one another in the longitudinal direction (X), an absorbing portion 42 extending along the longitudinal direction (X) and connected to the connecting portions 41, and a plurality of cotton fluffs 45 disposed on the absorbing portion 42. The absorbing portion 42 of each of the absorbing units 4 may be formed as parts that are each arcuate in shape. The connecting portions 41 and the absorbing portion 42 of each of the absorbing units 4 cooperate with the absorbent body 32 of the substrate 3 to define a plurality of aerated channels 44 each extending along the width direction.

With the absorbing units 4 being spaced apart along the width direction (Y), and the aerated channels 44, the sanitary napkin 2 has improved air permeability so as to prevent the generation of strange odors and promote comfort of use. Moreover, with the plurality of cotton fluffs 45 disposed on the absorbing portion 42, the sanitary napkin 2 may fit closely against a user's genitals so as to improve absorption and diffusion properties.

It should be noted herein that, two ends of each of the absorbing units 4 may be connected to the absorbent body 32 of the substrate 3 or may be configured to be spaced apart from the substrate 3.

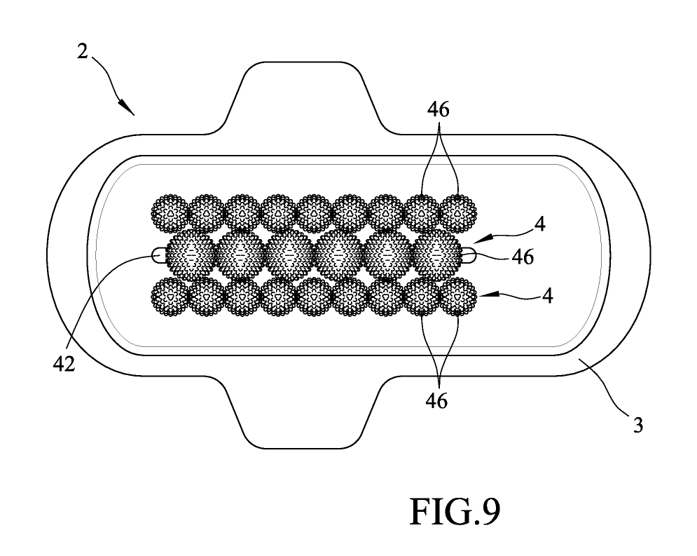

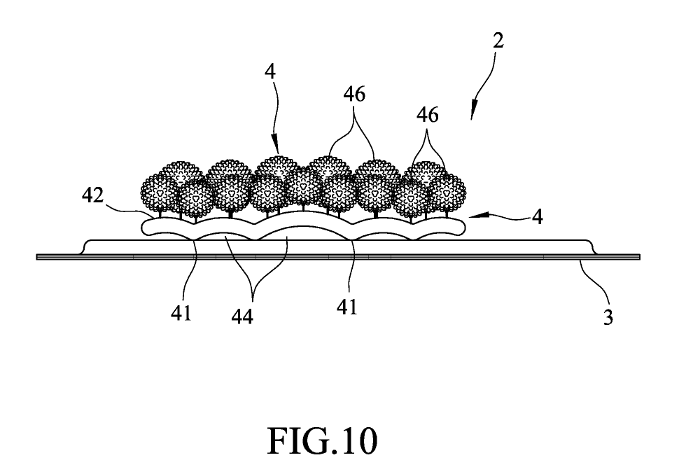

Referring to FIGS. 9 and 10, a third embodiment of a sanitary napkin 2 according to this disclosure is shown to be generally identical to the second embodiment, and only differs in that: each of the absorbing units 4 further includes a plurality of cotton fluff balls 46 disposed on the absorbing portion 42, and the diameters of the cotton fluff balls 46 on one of the absorbing units 4 are different from the diameters of the cotton fluff balls 46 on an adjacent one of the absorbing units 4.

This embodiment may achieve the same effects as the second embodiment. Since the diameters of the cotton fluff balls 46 on any two adjacent absorbing units 4 are different, there are many gaps among the cotton fluff balls 46, which improve air permeability so as to prevent the generation of strange odors and thereby promote comfort of use.

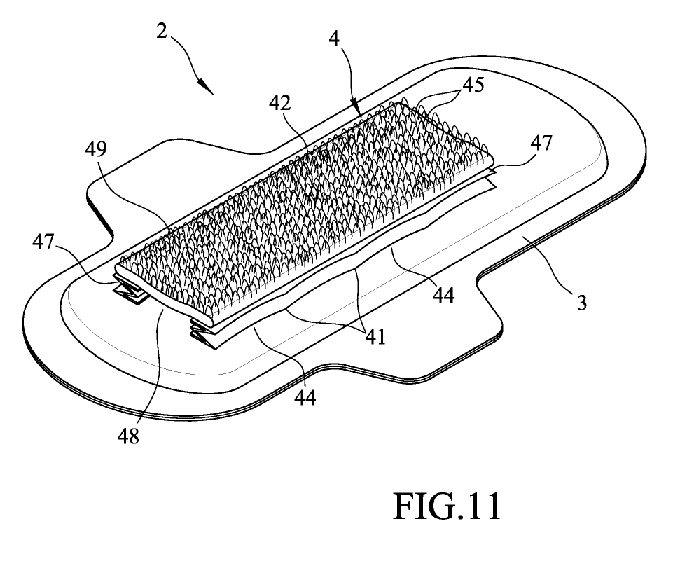

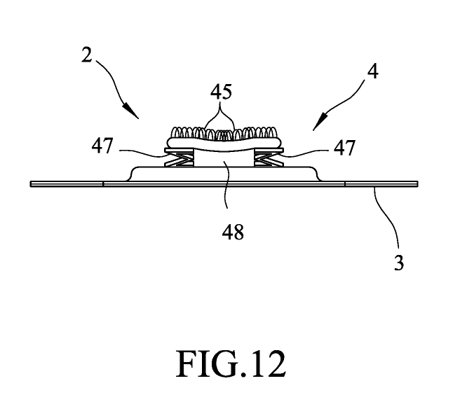

Referring to FIGS. 11 and 12, a fourth embodiment of a sanitary napkin 2 according to this disclosure is shown to be generally identical to the first embodiment, and only differs in that: the connecting portions 41 are arranged in two rows extending in the longitudinal direction (X), and the absorbing portion 42 has two pleated connecting parts 47 spaced apart from each other along the width direction (Y) and extending in the longitudinal direction (X), and an absorbing part (49). Each of the pleated connecting parts (47) interconnects the absorbing part (49) and a respective one of the two rows of the connecting portions (41). The absorbent body (32) of the substrate (3), the connecting portions (41), the pleated connecting parts (47), and the absorbing part (49) cooperatively define the aerated channels (44) each extending along the width direction (Y) and a ventilation channel (48) that extends along the longitudinal direction (X) and that is spatial communication with the aerated channels (44). This embodiment may achieve the same effects as the first embodiment, and also provide enhanced air permeability so as to promote comfort of use.

In summary, with the absorbing portion 42 which is connected to the absorbent body 32 of the substrate 3, as well as the aerated channels 44, the sanitary napkin 2 exhibits superior air permeability so as to prevent the generation of strange odors and thereby promote comfort of use.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A sanitary napkin comprising:
a substrate having a base layer and an absorbent body mounted protrudingly on said base layer, said substrate defining a longitudinal direction (X) and a width direction (Y) perpendicular to the longitudinal direction (X); and
at least one absorbing unit including a plurality of connecting portions connected to said absorbent body of said substrate and spaced apart among one another in the longitudinal direction (X), an absorbing portion extending along the longitudinal direction (X) and connected to said connecting portions, and a plurality of cotton fluffs disposed on said absorbing portion, said absorbent body of said substrate, said connecting portions and said absorbing portion cooperatively defining a plurality of aerated channels each extending along the width direction (Y).

2. The sanitary napkin of claim 1, wherein said absorbing unit is formed with a plurality of perforations extending through said absorbing portion and being disposed in fluid communication with said aerated channels.

3. The sanitary napkin of claim 1, further comprising a plurality of said absorbing units that are spaced among one another along the width direction (Y).

4. The sanitary napkin of claim 3, wherein each of said absorbing units further includes a plurality of cotton fluff balls disposed on said absorbing portion, and the diameters of said cotton fluff balls on one of said absorbing units are different from the diameters of said cotton fluff balls on an adjacent one of said absorbing units.

5. The sanitary napkin of claim 1, wherein said absorbing unit further includes a plurality of rows of cotton fluff balls disposed on said absorbing portion, and the diameters of said cotton fluff balls in one of said rows are different from the diameters of said cotton fluff balls in an adjacent one of said rows.

6. The sanitary napkin of claim 1, wherein said connecting portions are arranged in two rows each extending in the longitudinal direction (X), and said absorbing portion has two pleated connecting parts spaced apart from each other along the width direction (Y) and extending in the longitudinal direction (X), and an absorbing part, each of said pleated connecting parts interconnecting said absorbing part and a respective one of the two rows of said connecting portions, said absorbent body of said substrate, said connecting portions, said pleated connecting parts, and said absorbing part cooperatively defining said aerated channels and a ventilation channel that extends along the longitudinal direction (X) and that is spatial communication with said aerated channels.
